(19) United States
(12) Patent Application Publication
(10) Pub. No.: US 2007/0055365 A1
(43) Pub. Date: Mar. 8, 2007

(54) Stent with integrated filter

(75) Inventors: Roy K. Greenberg, Bratenahl, OH (US); John A. Deford, Richfield, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/412,465

(22) Filed: Apr. 27, 2006

Related U.S. Application Data
(60) Provisional application No. 60/676,099, filed on Apr. 28, 2005.

(51) Int. Cl. A61F 2/06

(52) U.S. Cl. 623/1.44

(57) ABSTRACT

A medical device that may be implanted intraluminally in a patient includes a stent and a filter combination. In one variation, the device includes a stent with a distal portion adapted to capture particles during the placement of the device in a stenosed region within a blood vessel. In another variation, the device includes a collapsible filter positioned within the distal lumen of the stent.

### RELATED APPLICATION

This application claims the benefit of priority to U.S. Application No. 60/676,099, filed Apr. 28, 2005, which is incorporated by reference into this application as if fully set forth herein.

### BACKGROUND OF THE INVENTION

A common problem associated with stent implantation is that the placement of the stent may cause materials or tissues (e.g., embolic material, atherosclerotic materials, plague, etc.) on the blood vessel wall to dislodge or break off. These loosened particles can cause serious damage when they enter the brain, lung, or other tissues and organs, resulting in stroke, pulmonary embolism, tissue damage and/or organ damage.

Various distal protection devices have been developed to capture and remove these dislodged materials during the stent placement procedure. Examples of various vessel filters, stents, and distal protection devices are disclosed in U.S. Patent Application Publication No. 2004/0111142 A1, titled “MICRO-POROUS MESH STENT WITH HYBRID STRUCTURE” by Rourke et al., published Jun. 10, 2004; U.S. Patent Application Publication No. 2004/0024416 A1, titled “IMPLANTABLE BRAIDED STROKE PREVENTING DEVICE AND METHOD OF MANUFACTURING” by Yodfat et al., published Feb. 5, 2004; U.S. Patent Application Publication No. 2004/0010307 A1, titled “IMPLANTABLE INTEGRAL DEVICE AND CORRESPONDING METHOD FOR DEFLECTING EMBOLIC MATERIAL IN BLOOD FLOWING AT AN ARTERIAL BIFURCATION” by Grad et al., published Jan. 15, 2004; U.S. Patent Application Publication No. 2003/0125801 A1, titled “IMPLANTABLE STROKE TREATING DEVICE” by Yodfat et al., published Jul. 3, 2003; U.S. Pat. No. 6,843,802 B 1 titled “DELIVERY APPARATUS FOR A SELF EXPANDING RETRACTABLE STENT” issued to Villalobos et al., dated Jan. 18, 2005; U.S. Pat. No. 6,695,813 B1 titled “EMBOLIC PROTECTION DEVICES” issued to Boyle et al., dated Feb. 24, 2004; U.S. Pat. No. 6,685,738 B2 titled “BRAIDED ENDOLUMINAL DEVICE HAVING TAPERED FILAMENTS” issued to Chouinard et al., dated Feb. 3, 2004; U.S. Pat. No. 6,673,089 B1 titled “IMPLANTABLE STROKE TREATING DEVICE” issued to Yassour et al., dated Jan. 6, 2004; U.S. Pat. No. 6,645,222 B1 titled “PUNCTURE RESISTANT BRANCH ARTERY OCCLUSION DEVICE AND METHODS OF USE” issued to Parodi et al., dated Nov. 11, 2003; 6,592,616 B1 titled “SYSTEM AND DEVICE FOR MINIMIZING EMBOLIC RISK DURING AN INTERVENTION PROCEDURE” issued to Stack et al., dated Jul. 15, 2003; U.S. Pat. No. 6,582,396 B1 titled “PUNCTURE RESISTANT BALLOON FOR USE IN CAROTID ARTERY PROCEDURES AND METHODS OF USE” issued to Parodi, dated Jun. 24, 2003; U.S. Pat. No. 6,443,971 B1 titled “SYSTEM FOR, AND METHOD OF, BLOCKING THE PASSAGE OF EMBOLI THROUGH A VESSEL” issued to Boylan et al., dated Sep. 3, 2002; U.S. Pat. No. 6,267,777 B1 titled “VASCULAR FILTER CONVERTIBLE TO A STENT AND METHOD” issued to Bosma et al., dated Jul. 31, 2001; U.S. Pat. No. 6,241,746 B1 titled “VASCULAR FILTER CONVERTIBLE TO A STENT METHOD” issued to Bosma et al., dated Jun. 5, 2001; U.S. Pat. No. 6,042,598 titled “METHOD OF PROTECTING A PATIENT FROM EMBOLIZATION DURING CARDIAC SURGERY” issued to Tsugita et al., dated Mar. 28, 2000; U.S. Pat. No. 6,027,520 titled “PERCUTANEOUS CATHETER AND GUIDEWIRE HAVING FILTER AND MEDICAL DEVICE DEPLOYMENT CAPABILITIES” issued to Tsugita et al., dated Feb. 22, 2000; U.S. Pat. No. 6,013,085 titled “METHOD FOR TREATING STENOSIS OF THE CAROTID ARTERY” issued to Howard, dated Jan. 11, 2000; U.S. Pat. No. 5,910,154 titled “PERCUTANEOUS CATHETER AND GUIDEWIRE HAVING FILTER AND MEDICAL DEVICE DEPLOYMENT” issued to Tsugita et al., dated Jun. 8, 1999; U.S. Pat. No. 5,800,525 titled “BLOOD FILTER” issued to Bachinski et al., dated Sep. 1, 1998; U.S. Pat. No. 4,655,771 titled “PROSTHESIS COMPRISING AN EXPANSIBLE OR CONTRACTILE TUBULAR BODY” issued to Wallsten, dated Apr. 7, 1987; each of which is incorporated herein by reference in its entirety.

Typically, a vessel filter positioned distal to the stent placement site is utilized to capture the dislodged materials. For example, a filter is first deployed downstream of the stenosed region by passing a filter via a low-profile delivery catheter (e.g., 4 Fr) through the stenosed region. Next, a stent is delivered to the stenosed region and deployed. The embolic materials breaking free from the blood vessel wall as a result of balloon or stent expansion are then captured by the distally positioned filter. However, various complications and limitations are associated with this procedure. For instance, because the stent and the distal protection device each require its own deployment mechanism, the placement of the distal protection device complicates the stent implant procedure and increases the probability of complications. Once the stent is positioned in place, the distal protection device will need to be removed though the lumen of the deployed stent without disturbing the positioning of the stent, which may prove to be quite difficult. In addition, in most designs, proper spacing needs to be maintained between the distal protection device and the stent in order for each to function properly, further increasing the complexity of the stent implant procedure. In some cases, patients are ineligible for the procedure due to lack of adequate spacing at the stent implant site.

### BRIEF SUMMARY OF THE INVENTION

One aspect of the invention includes a stent with an integrated filtering mechanism. The implant procedure may be accomplished with the deployment of a single device, eliminating the need for spacing between devices. In one variation, the stent/filter device includes a distal portion configured to serve as a filter, while the proximal portion is adapted to act as the stent. For example, the device can include a self-expanding stent with a lattice structure where the distal portion of the stent is configured to serve as a filter. The distal portion may be configured with pores that are sized to allow blood flow, while at the same time preventing clots or emboli from passing through. Preferably, each of the pores is at least 10 micron in diameter. More preferably, each of the pores is at least 20 micron in diameter. In another variation, a stent is configured with means for filtering blood flow. The filtering mechanism includes, but not limited to, polymer layer with pores, mesh layer, wire mesh, polymer mesh, metallic mesh, fabric mesh, web, net, etc.

During the device deployment procedure, the distal portion is deployed first, forming a filter distally positioned relative to the stenosed region, trapping any embolic material and catching any subsequent material that breaks free as a result of deployment of the proximal portion of the device. Once the complete device is deployed, a balloon may be introduced into the lumen of the device to further expand the diameter of the vessel at the stenosed region. In another approach, a delivery system with an integrated balloon may be utilized to deploy the dual purpose device. In this approach, the distal filter portion of the device is again deployed first, followed by expansion of the stent portion of the device utilizing the balloon on the delivery system.

In another variation, the distal portion of the stent/filter device may be configured with a nitinol tube having pore sizes that prevent clots or debris from passing therethrough, while the proximal portion may be configured as a standard self-expanding stent with a lattice structure. In yet another design, a typical stent structure is covered with a biocompatible polymer (e.g., expanded polytetrafluoroethylene (ePTFE), etc.) around its circumferential surface, and pores are provided on the biocompatible polymer layer. In one variation, the distal portion polymer covering is configured with pores to allow blood flow, while at the same time having the ability to capture emboli or other large particles. In another variation, both the distal and proximal portions of the polymer covering are configured with pores. The pores at the distal portion may be smaller than the pores at the proximal portion such that fragments of the lesion dislodging from the stenosed region may be captured by the distal portion of the device. The pores at the proximal portion may be larger such that stenting over a bifurcation does not impede blood flow into or from the branching vessel. In another design, a porous polymeric covering is only provided over the distal portion of the stent body.

In another variation, an integrated device is provided to permit the user to perform the stent/filter placement procedure through a single catheter insertion, and avoid the need to exchange balloon catheters or other medical instruments to deploy the stent/filter. In one example, a pre-dilatation balloon, a stent/filter device, and a post-dilatation balloon are incorporated into a single delivery apparatus, such that the entire procedure can be performed with a single catheter insertion. One variation of the integrated device allows the user to insert a single device carrying all the functional elements into the implantation site to perform the following steps: deploy the distal portion of the stent/filter; expand the lesion (e.g. using a balloon, etc.); deploy the rest of the stent/filter device; further expand the stent/filter to conform to the vessel (e.g. using a balloon). As one of ordinary skill in the art having the benefit of this disclosure would appreciate, variations of the implantation apparatus may be configured such that one or more of these steps can be perform simultaneously.

In another aspect of the invention, a collapsible filter is positioned at the distal end of a stent or within the lumen of the stent to provide the protective function. The distal portion of the stent along with its integrated filter may be deployed first to capture particles within the blood stream. After the complete stent is positioned in place, an optional procedure may be performed to disable or remove the integrated filter.

These and other embodiments, features and advantages of the present invention will become more apparent to those skilled in the art when taken with reference to the following more detailed description of the invention in conjunction with the accompanying drawings that are first briefly described.

### BRIEF DESCRIPTION OF THE DRAWINGS

FIG. 1A illustrates one variation of a stent/filter device with a porous filter integrated on the distal portion of the stent body. The stent/filter device is shown in an expanded condition.

FIG 1B is a partially transparent view of the stent/filter device of FIG. 1A showing the flexible metal wire mesh lattice forming the stent.

FIG. 2 is a cross-sectional view illustrating the stent/filter device of FIG. 1A positioned within a blood vessel.

FIG. 3 illustrates another variation of a stent/filter device comprising a porous polymeric layer positioned over the tubular stent structure. The stent/filter device is shown in an expanded condition.

FIG. 4 illustrates another variation of a stent/filter device where the distal portion of the device is configured with small pores to serve as a filter, and the proximal portion of the device is configured with larger pores. The larger pores may be configured to permit perfusion of perforating vessels or vessel branches.

FIG. 5 illustrates another variation of a stent/filter device in which the porous polymer layer is implemented only on the distal portion of the stent.

FIG. 6 illustrates yet another variation of a stent/filter device having two segments of flexible lattice structures in which the distal portion wire mesh is configured with higher density then the proximal portion wire mesh. The distal and proximal lattice may be of the same or different types of material.

FIG. 7 illustrates a configuration of a stent/filter device positioned over a targeted region within a blood vessel.

FIG. 8 illustrates another configuration of a stent/filter device. In this example, a deployment apparatus with an integrated balloon is utilized for the deployment of the stent/filter device.

FIG. 9 illustrates another configuration for deploying a stent/filter device. In this example, an inflatable balloon is implemented to facilitate the initial filter deployment.

FIG. 10 illustrates another variation of a stent/filter device having a filter positioned over the distal opening of a tubular stent.

FIG. 11 illustrates another variation of a stent/filter device that has a low profile collapsible filter is positioned at the distal end of a stent.

FIG. 12 illustrates yet another variation of a stent/filter device comprising a filter positioned within the lumen of a stent.

FIG. 13A illustrates one variation of an integrated stent/filter delivery apparatus. This configuration allows the user to introduce a stent/filter and two dilatation balloons with a single delivery catheter.

FIG. 13B illustrates the delivery apparatus of FIG. 13A with the delivery catheter partially retracted, and one of the balloons partially inflated to expand the distal portion of the stent.

FIG. 13C illustrates the delivery apparatus of FIG. 13B with the delivery catheter further retracted to expose the complete stent and the pre-dilatation balloon. The pre-dilatation balloon, as shown, can be inflated to dilate the stenosed region.

FIG. 14 illustrates one variation of a delivery catheter including a balloon position over the shaft of the elongated catheter body. The balloon is shown in an inflated condition.

FIG. 15 illustrates one approach in utilizing a delivery catheter with an integrated balloon to deliver a stent into a vessel.

FIG. 16 illustrates another variation of a delivery apparatus. In this variation the apparatus comprises a stent positioned over a balloon catheter, and the balloon catheter/stent unit is inserted within a delivery catheter with an integrated balloon. Optionally the apparatus may further include an introducer tubing to facilitate the placement of the delivery catheter, along with the stent and the balloon catheter secured within the lumen of the delivery catheter.

FIG. 17 illustrates a deployment of a stent/filter. The delivery catheter with an integrated balloon is partially retracted to expose the distal portion of the stent. The balloon catheter positioned within the lumen of the stent is partially inflated to expand the distal portion of the stent. Once the balloon catheter is deflated, the distal portion of the stent remains expanded against the wall of the blood vessel to serve as a filter.

FIG. 18 illustrates yet another variation of a delivery apparatus. In this variation, the delivery catheter is configured with two parallel lumens extending along the length of the catheter. The first lumen is configured to provide a fluid conduit for inflating the integrated balloon at the distal end of the delivery catheter. The second lumen houses a balloon catheter for expanding the stent.

FIG. 19 illustrates one variation of a stent deployment apparatus including a collapsible filter positioned at the distal end of the delivery apparatus. In this particular design, the filter comprises a collapsible cone-shaped body positioned with the tip of the cone towards the stent, such that after the stent is deployed, the filter can be easily retracted within the delivery catheter.

### DETAILED DESCRIPTION OF THE INVENTION

The following detailed description should be read with reference to the drawings, in which identical reference numbers refer to like elements through out the different figures. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

It is to be understood that unless otherwise indicated, the preferred embodiments need not be limited to applications in humans. As one of ordinary skill in the art would appreciate, variations of the preferred embodiments may be applied to other animals as well. Moreover, it should be understood that embodiments of the present invention may be applied in combination with various catheters, guidewires, tubing introducers or other stent deployment devices, for implantation of the stent/filter device in a hollow body organ within a patient's body.

Implantation of the stent/filter device within a carotid artery is used herein as an example application of the stent/filter device. In light of the disclosure herein, one of ordinary skill in the art would appreciate that variations of the stent/filter device may be applicable for placement in various ducts, blood vessels, hollow body organs or elongated cavities in a mammalian body. The stent/filter described herein may be implemented for capturing particles other than blood clots.

Furthermore, one of ordinary skill in the art having the benefit of this disclosure would appreciate that variations of the preferred embodiments may be applicable in various medical conditions including but not limited to carotid stenosis, obstructions of the urinary tract, renal artery obstructions, and kidney obstructions. The preferred embodiments may also be implemented along with various vascular surgery procedures (e.g., placement of vascular grafts within the coronary system of the patients who need coronary bypass or removal of vascular lesions, etc.) for capturing debris generated during the vascular procedure and/or during post surgical recovery.

It must also be noted that, as used in this specification and the appended claims, the singular forms “a,” “an” and “the” include plural referents unless the context clearly dictates otherwise. Thus, for example, the term “filter” is intended to mean a single filter or a combination of filters, “a polymer” is intended to mean one or more polymers, or a mixture thereof. Furthermore, the words “proximal” and “distal” refer to directions closer to and away from, respectively, a physician implanting the stent, with the distal end placed downstream within a fluid channel, and the proximal end placed upstream where fluids enters the lumen of the stent. Thus, once the stent/filer device is deployed within a blood vessel, the blood flows into the device through the proximal opening and exits the device through the distal opening. A “pore” is intended to mean a hole, an orifice, or an opening, admitting a flow of a liquid through a layer or object. Pores are sized to allow blood flow there through, unless the context clearly dictates otherwise. In one variation, pores are configured to allow blood flow, while at the same time preventing clots or emboli from passing through. Preferably, each of the pores is at least 10 micron in diameter.

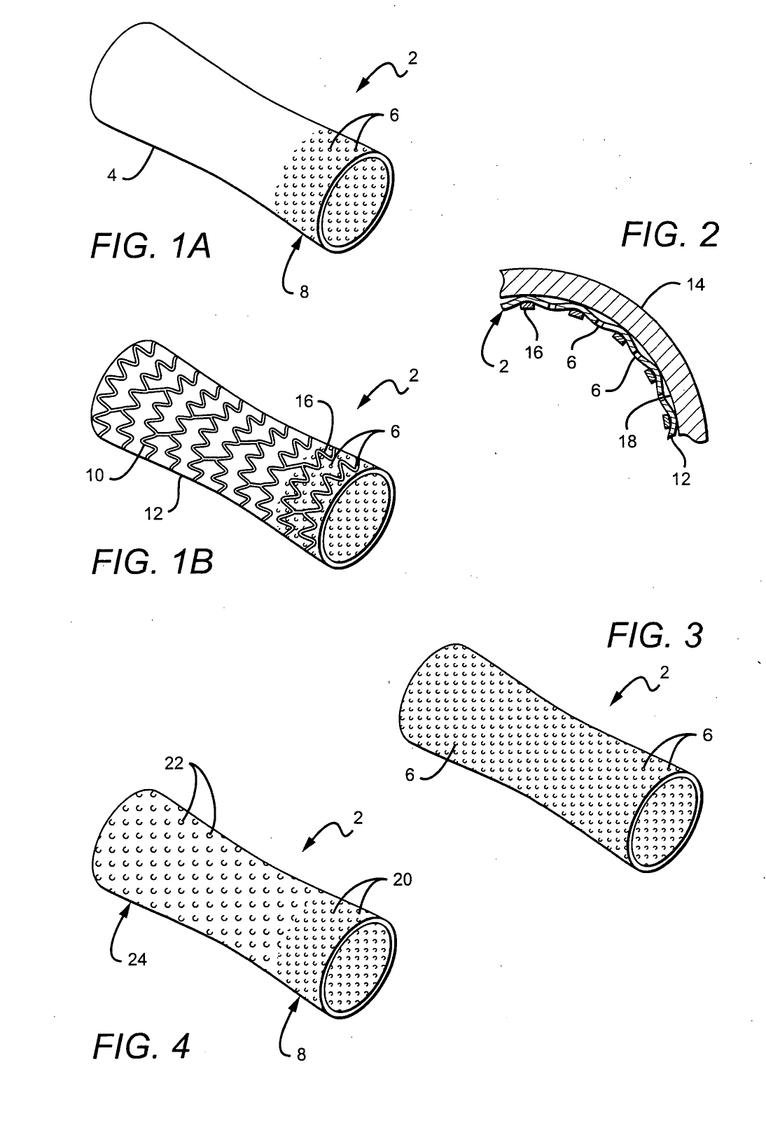

In one aspect, the stent/filter device 2 includes a stent 4 with a plurality of pores/holes/orifices 6 located on the distal portion 8 of the stent body. An example is shown in FIG. 1A. The device 2 may be designed with pores/holes/orifices distributed over the distal portion 8 only. In another variation, the pores/holes/orifices may be distributed along the length of the stent. In another design, the complete stent is covered with a flexible material (e.g., a polymeric layer, etc.), and pores may be distributed over the entire covering or the pores may be limited to the distal portion of the device. Preferably, these pores/holes/orifices are large enough to permit blood to flow through. In another design, only the distal portion of the stent is covered with a porous polymeric layer, while the rest of the stent lattice is exposed without a covering. In addition, pores may also be created on the stent by placing a thin mesh of material over the stent structure. The thin mesh of material may be metallic, metallic alloy, polymer or other suitable materials. In another design, polyurethane is nano-spun onto the metallic alloy forming the stent structure. Polymeric materials may also be weaved onto the circumferential surface of the stent to form pores along the length of the stent. Other biocompatible materials or fibers may also be woven or knitted onto the lattice of the stent to form pores or filter over the circumferential surface of the stent.

These materials and coatings (e.g., polymeric layer, mesh, etc.), which are utilized to cover the circumferential surface of the stent body, may incorporate an active agent to facilitate healing of tissues and/or provide other therapeutic benefits. For example, the material and/or coating covering the stent lattice may be utilized as a reservoir to hold therapeutic agents (e.g., antithrombogenic agent, anti-inflammatory agent, antibacterial agent, anti-viral agent, etc.), which can be delivered to the implantation site during and/or after the device delivery. In one variation, a polymeric layer covering the circumferential surface of the stent lattice structure includes a pharmaceutical to reduce inflammation, promote vessel healing, and/or prevent restenosis. In another variation, a biological material (e.g., monoclonal antibodies, growth factors, cells, stem cells, cartilage, etc.) is incorporated within the polymeric layer or mesh layer covering the lattice of the stent. The therapeutic agents and/or biological materials may be incorporated, integrated, infused or otherwise loaded into the polymeric layer or mesh layer.

In yet another design, the stent comprises two or more segments of flexible metals wire mesh each having a different distribution/density pattern. For example, at the distal portion, the lattice wire mesh may be densely distributed to form small pores, while the proximal portion has a distribution pattern similar to one of the traditional stent lattice. One of ordinary skill in the art having the benefit of this disclosure would appreciate that various other methods that are well known to one of ordinary skilled in the art may be utilized to form pores over a stent.

In one variation, the diameters of the pores at the proximal portion of the stent can be from about 50 micron to about 100 micron. In another variation, the diameters of the pores can be from about 70 micron to about 100 micron. The pores may be uniform in size or they may be variable. The pores may also be of circular or oval in shape. In another design, each of the pores has a cross-sectional area from about 1900 microns-squared to about 10000 microns-squared.

The stent/filter structure may be configured with different shapes and sizes depending on the particular application. For example the stent/filter device may be tapered to conform to the variability in vessel diameter of a carotid artery, where one may expect a larger diameter at the proximal portion. The stent/filter device may also be designed with enough flexibility to conform to the variation in vessel lumen surface along the length of the vessel. Other variations the stent/filter device may utilize the segmented technology well known in the stent design/manufacturing industry. For example, the stent/filter device may comprise one or more biodegradable sections between the stent rings.

In one example, as illustrated in FIG. 1B, the stent/filter device 2 includes a flexible metallic stent 10 with its circumferential surface covered by a polymeric layer 12. The polymeric layer 12 may include one or more of the various biocompatible polymers (e.g., ePTFE, PTFE, urethane, polyurethane, silicone, nylon, etc.) that are suitable for placement over a stent lattice. The stent 10 may be self-expandable. That is, once placed in the target environment, the stent changes its physical shape so as to expand. The distal portion 8 of the polymeric layer 12 may include a plurality of pores 6, such that the distal portion 8 of the device may serve as a filter during the deployment of the device 2.

FIG. 2 is a cross-sectional view illustrating a portion of an expanded stent/filter 2 compressing against the wall 14 of a blood vessel. In this particular design, the material (e.g., polymer, metal, metal alloy, etc.), which forms the stent lattice 16, applies a radial force through the polymeric layer 12 to counter the compression of the blood vessel wall 14. The pores 6 on the polymeric layer 12 may allow blood to pass through the polymeric layer 12 and perfuse the endothelial cells which forms the inner wall 18 of the blood vessel.

FIG. 3 illustrates one variation, where pores 6 are distributed along the complete length of the stent/filter device 2. FIG. 4 shows another variation of the stent/filter device 2 having small pores 20 in the distal portion, while the pores 22 on the proximal portion 24 are larger. For example, the pores 20 in the distal portion 8 may be from about 50 micron to about 100 micron, while the pores 22 in the proximal portion 24 may be from about 100 micron to about 200 micron. A stent/filter device with large proximal pores 22 may be particularly useful in applications where the device 2 is to be placed over a bifurcating segment of the blood vessel. For example, there may be a series of smaller vessels branching off the carotid artery. When a stent is placed inside the carotid artery, it may be beneficial to avoid occluding these side branches. Occlusion of the branching vessel may cause ischemia in the tissue or organs being perfused by the branching vessel. Therefore, when a stent/filter device is placed over a branching vessel, it may be desirable to provide pores to permit blood flow into the branching vessel. However, small pores may occlude over time due to thrombus formation. Thus, large pores may be preferable in the proximal portion of the device; while small pores may be utilized at the distal portion to ensure that a significant amount of atherosclerotic materials, which have dislodged from the stenosed region, are captured by the device. In one variation, the pore size is configured to provide a path for vessel healing and/or re-endothelialization.

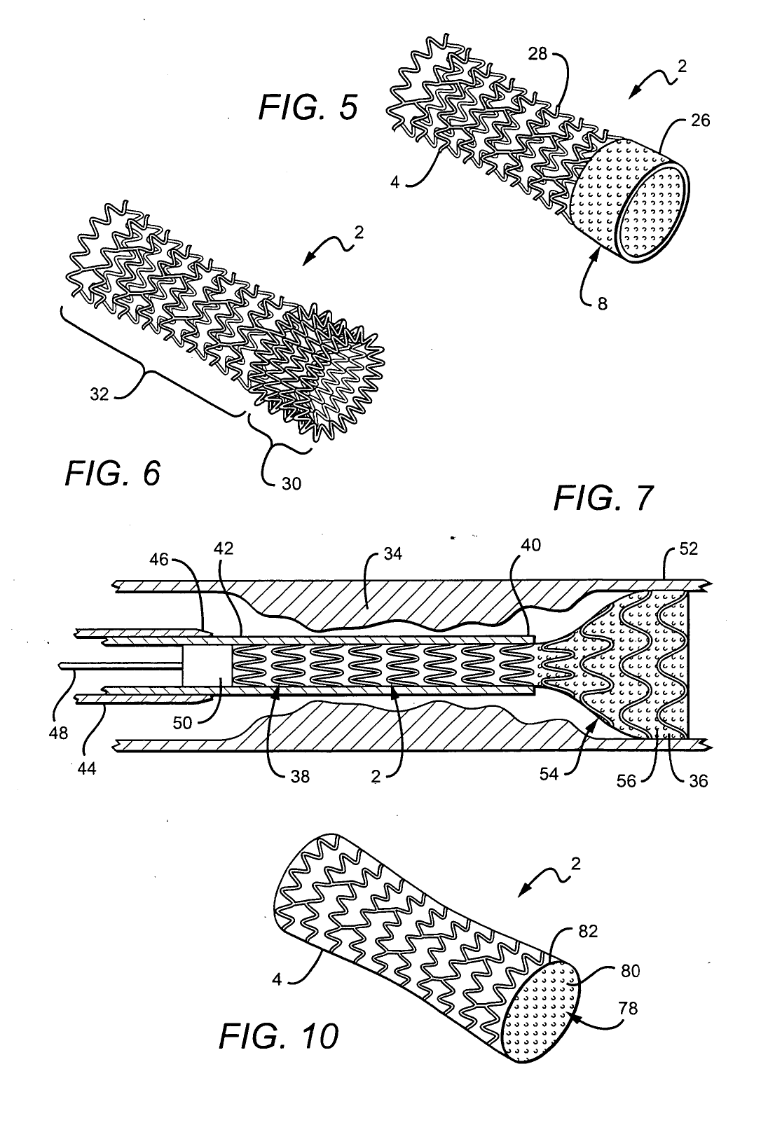

FIG. 5 illustrates one variation where only the distal portion 8 of the stent 4 is covered with a porous polymeric layer 26. The lattice 28 forming the stent may comprise nitinol or other self-expanding material, such that when the device 2 is deployed inside a blood vessel, the stent 4 can self-expand. The distal portion 8 with the porous covering may be deployed first to provide distal protection.

FIG. 6 illustrates yet another variation in which the stent/filter device 2 includes two segments 30, 32 of different materials. The distal portion 30 may be fashioned in the form of a nitinol tube with pore sizes configured to prevent clots from passing therethrough, while the proximal portion 32 may be fashioned as a standard stent with a lattice structure. In this example, the distal portion 30 includes nitinol wire mesh that form a dense distribution pattern when deployed. The nitinol material is configured such that the distal portion will self-expand. The dense wire mesh distribution of the circumferential surface forms a filter having small pores to capture blood clots. The proximal portion 32 comprises a lattice that is loosely distributed (in comparison to the distal portion) over the circumference of the device. The proximal portion 32 may be configured to be either self-expandable or balloon-expandable. In a self-expandable design, after the filter has been deployed, a balloon may be subsequently introduced to further expand the deployed stent and compress the stent against the blood vessel wall. In another variation, the complete device comprises a self-expanding nitinol material. However, the distal portion is configured with a dense lattice structure, while the proximal portion is configured with a dispersed lattice structure.

Referring to FIG. 7, an exemplary approach in deployment of the stent/filter device 2 is illustrated. The stent/filter device 2 is to be positioned over a targeted (e.g., stenosed) region 34 with the filter portion 36 of the device located distally (i.e., downstream from blood flow) of the stenosed region 34. The filter portion 36 of the device 2 is deployed first to provide distal protection. The filter 36 may trap any embolic material and catch any subsequent material that breaks free as a result of deployment of the proximal portion 38 of the device 2. After the filter portion 36 is deployed, the proximal portion 38 of the device 2 is deployed over the stenosed region 34 (e.g., the atherosclerotic materials) on the vessel wall 52.

In the example shown in FIG. 7, a self-expanding stent/filter device 2 is inserted into the distal end 40 of a delivery catheter 42 prior to delivery. In one variation, the delivery catheter's diameter is about 6 French or smaller. An introducer sheath 44 assisted by a guidewire is inserted into the vascular system and advanced towards the target (e.g., stenosed) region. Once the distal end 46 of the introducer sheath 44 is positioned at a location proximal of the stenosed region 34, the guidewire is removed, and the delivery catheter 42 carrying the stent/filter device 2 is inserted into the introducer sheath 44 and advanced towards the stenosed region 34. Eventually, the distal tip 40 of the delivery catheter 42 will exit the tip 46 of the introducer sheath 44 and then pass through the stenosed region 34. With the tip 40 of the delivery catheter 42 positioned beyond the stenosed region 34 and a pusher wire 48 with its pusher pad 50 keeping the stent/filter device 2 in place, the user can slowly retract the delivery catheter 42 to expose the distal portion 54 of the stent/filter device 2. In this approach, the stent/filter 2 expands radially, and movements in the distal/proximal direction along the length of the vessel 52 may be minimized, thus preventing the device 2 from causing abrasion to the vessel wall 52. The distal portion 54 of the stent/filter device 2, which can include a self-expanding material, expands outward and forms a filter 36 downstream from the stenosed region 34, as shown in FIG. 7. The pores 56 on the filter 36 allow the fluids in the blood stream to pass through, but prevent any emboli or large particles from passing through.

With the distal filter portion 54 of the stent/filter device 2 deployed, the user may then continue to withdraw the delivery catheter 42 and deploy the proximal portion 38 of the stent/filter device 2 over the stenosed region 34. As the stent/filter device 2 expands and compresses against the vessel wall 52, particles that are captured by the distal filter portion 36 of the device are pressed against the vessel wall 52 and removed from circulation. Once the complete stent/filter device 2 has been deployed, the user may withdraw the delivery catheter 42 along with the pusher wire 48. To further break apart the atherosclerotic plaque and/or expand the stenosed region 34 of the vessel, the user may introduce a balloon through the introducer sheath 44 and into the lumen of the deployed stent/filter device 2. The balloon is then inflated inside the lumen of the stent/filter device 2. The expansion of the balloon forces the stent/filter device 2 to expand and further compress against the vessel wall 52 at the stenosed region 34. Once the procedure is completed, the balloon along with the introducer sheath 44 may be withdrawn from the patient's body. In a different deployment approach, the filter 36 is deployed by pushing the stent/filter device 2 out of the delivery catheter 42 instead of retracting the delivery catheter 42.

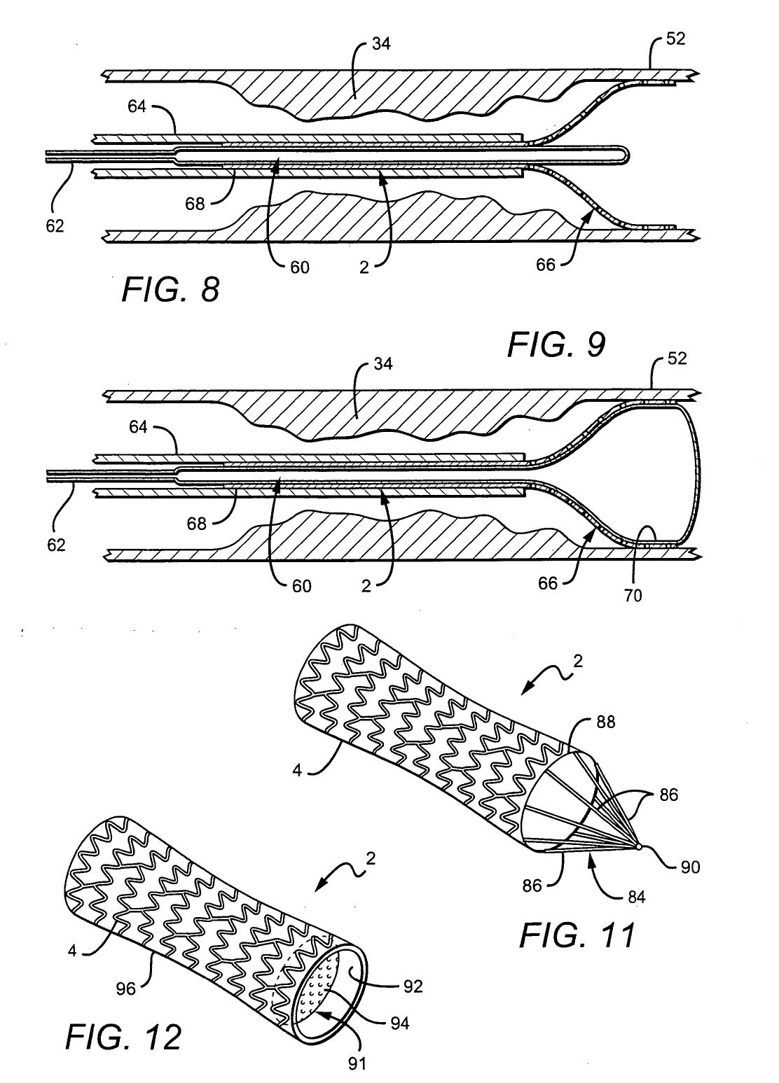

In another example, shown here in FIGS. 8 and 9, a delivery system with an integrated balloon is utilized to deliver the stent/filter device. The balloon may be a compliant or non-compliant balloon. Other means that are well known to one of ordinary skill in the art for deploying a stent may also be implemented to expand the stent. For example, the means for expanding the stent may include one or more of the following: a diaphragm, a compliance balloon, a non-compliance balloon, a mechanical expansion mechanism, etc. The stent/filter device 2 is positioned and/or compressed over the balloon 60 on a balloon catheter 62, and placed inside the lumen of a delivery catheter 64. The integrated balloon and stent/filter 2 unit is then introduced into the stenosed region 34 through an introducer sheath. Once the delivery catheter 64 loaded with the stent/filter 2 and the balloon 60 is positioned in place, the user may withdraw the delivery catheter 64 to expose the distal portion 66 of the stent/filter device 2, allowing it to expand as shown in FIG. 8. As the user continues to retract the delivery catheter 64, the proximal portion 68 of the stent/filter 2 will be exposed. If the proximal portion 68 of the stent/filter 2 also includes a self-expanding material, then the stent/filter device 2 will expand into contact with the stenosed region 34. Alternatively, the balloon 60 can be inflated to expand the proximal portion 68 of the stent/filter device 2. Once the stent/filter device 2 is expanded over the stenosed region 34, whether through self-expansion or balloon inflation, the user can inflate the balloon 60 to further dilate the stenosed region 34.

In another variation, as the delivery catheter 64 is withdrawn and the distal portion 66 of the stent/filter device 2 is exposed, the balloon 70 can be inflated generally simultaneously. The expansion of the balloon 70 forces the distal portion 66 of the stent/filter device 2 to expand toward the vessel wall 52, as shown in FIG. 9. The stent/filter device 2 may be self-expandable or balloon-expandable. As the user continues with the withdrawal of the delivery catheter 64, the balloon 70 is further inflated to expand the rest of the stent/filter device 2. A non-compliant balloon may be utilized in this application. In another variation, a compliant balloon is utilized in the procedure illustrated in FIG. 9.

In another approach, once the distal portion of the stent/filter device is deployed, a balloon is inserted alongside the partially deployed stent/filter device. The balloon is inflated to expand the stenosed region. After the stenosed region has been expanded, the user can then deflate and retract the balloon. The proximal portion of the stent is then deployed over the stenosed region.

In another aspect, the stent/filter device 2 may include a stent 4 with a filter 78 integrated at the distal opening 82 of the stent 4. In one example, the device 2 may include a stent 4 with a porous polymeric film 80 covering the distal opening 82 of the stent 4, as shown in FIG. 10. The stent 4 may be either self-expandable or balloon-expandable. Immediately after the device 2, is deployed, suction may be applied through the delivery catheter or the introducer sheath to remove any particles captured by the filter. The user may then have the option of dismantling the filter 78. For example, the user may introduce a flexible rod having a tip configured to tear apart the porous polymeric film. The porous polymeric film may comprise a biodegradable material such that the shredded strips of polymeric film may disintegrate over time. In another example, the filter may include a biodegradable polymer, and after deployment, the filter is left in place and allowed to degrade over time. In yet another example, a flexible rod having a heating element at the distal tip is introduced to melt the filter away. The filter may comprise a biodegradable porous polymeric film having a low melting point, so that the user can easily dismantle the filter by melting it. In one variation, the biodegradable porous polymeric film (e.g., polycaprolactone, etc.) has a low melting point between about 45 degree Celsius to about 85 degree Celsius.

In another variation, the stent/filter device 2 may include a low profile collapsible filter 84 attached to the distal end of a stent. In one example, illustrated in FIG. 11, the filter 84 comprises a plurality of thin filaments 86 connected to the distal end 88 of the stent 4 to form a cone-shaped filter 84. The filter 84 may be configured such that after the stent/filter is implanted, the user can easily disable the filter 84. For example, the components of the filter 84 may be disassembled and pushed outward against the wall of the blood vessel. In one variation, a connecting member 90 at the distal tip of the cone-shaped filter 84 may be attached to one of the filaments 86 firmly, while attached to the other filaments loosely. A flexible rod with a blunt distal end may be introduced to force the disengagement of the connecting member 90 from the filaments and allowing the filaments 86 to disperse. In another variation, the connecting member 90 holding the filaments 86 together may comprise a biodegradable polymer having a low melting point, such that a heating element can be introduced to disassemble the filter 84. The plurality of filaments 86 may include a biodegradable polymer such that after they are disconnected from each other, the filaments 86 would degrade and disintegrate over time.

In another aspect, the stent/filter may include a collapsible filter 91 positioned within the lumen 92 of a stent 4. FIG. 12 illustrates one example, where the device 2 may be a porous polymeric film 94 attached within the lumen of a stent 4. The circumferential surface 96 of the stent can also be covered by a polymeric material. The filter 91 within the lumen of the stent may be shredded or otherwise destroyed after the completion of the implant procedure. The porous polymeric film 94, which forms the filter 91, may be biodegradable and/or have a low melting point. In other variations, the filter 91 comprises filaments, a mesh, or a net. Alternatively, variations of the filter may be implemented at the distal portion of the stent to form a stent/filter device.

In applications where the stent/filter device may have a biodegradable filter and the user chose not to dismantle the filter immediately after the procedure, suction may be applied to remove any particles captured within the filter during the implantation of the device. The filter is then left on the implanted device to provide protection against thrombus for a period of time until the filter eventually disintegrates due to natural degradation. The captured thrombus may lyse and then pass-through the filter, such that they do not cause harm to the patient. The filter will disintegrate over time, such that it will provide protection during the critical period of time immediately after the implant procedure, while there would be no need for additional surgical intervention to remove the filter later.

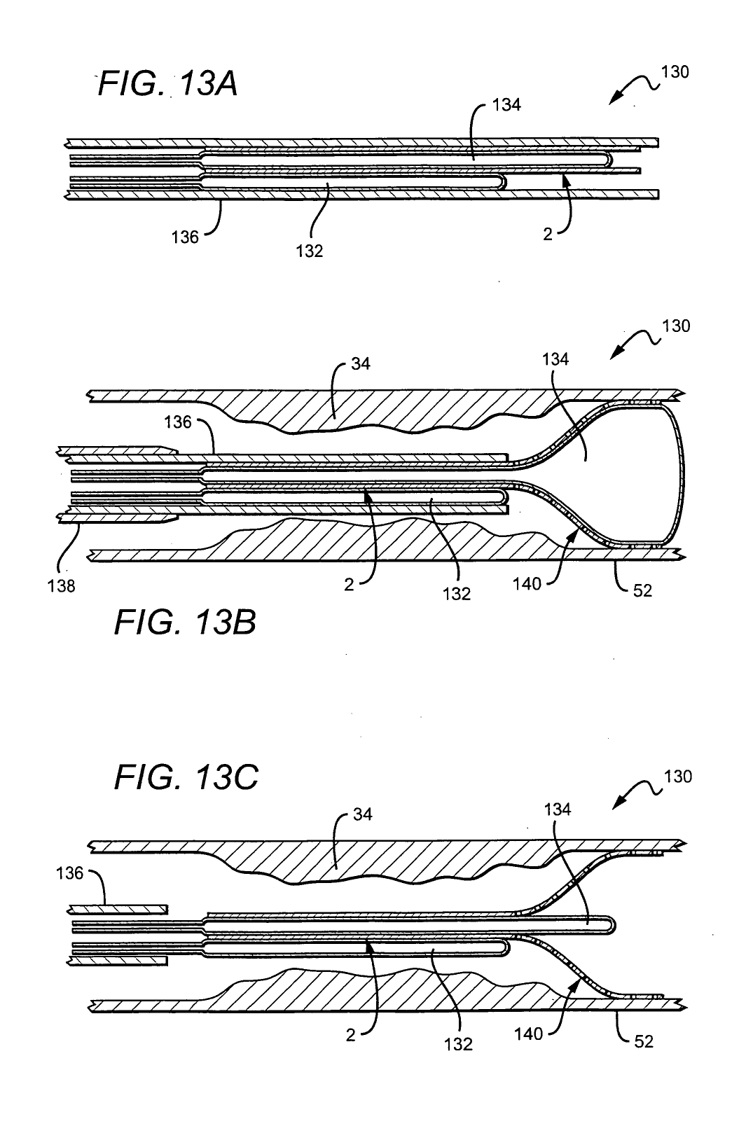

In another aspect, as shown here in FIGS. 13A-C, an integrated deployment apparatus is provided to allow the user to perform the stent/filter placement through a single catheter insertion, and avoid the need to exchange the expansion mechanisms in order to expand the stent and/or dilate the blood vessel. In one variation, the apparatus 130 may include a first expansion mechanism 132 for dilating the stenosed region of the blood vessel, while a second expansion mechanism 134 is configured for deploying the stent/filter 2. Various means for dilating and/or expanding a segment of a blood vessel may be implemented. For example, the means for dilating and/or expanding may include one or more of the following: a diaphragm, a compliance balloon, a non-compliance balloon, a mechanical expansion mechanism, etc. The expansion mechanism may be positioned on various locations on the deployment apparatus.

In one design variation, as shown in FIG. 13A, each of the expansion mechanisms (referenced here as 132, 134) may include a balloon catheter. The first balloon catheter 132, positioned between the device 2 and the delivery catheter 136, is configured to serve as a pre-dilatation balloon. The pre-dilatation 132 balloon may be inflated to break apart plaques at the stenosed region prior to the deployment of the device 2. The second balloon catheter 134, positioned within the lumen of the device 2, is configured to serve as the post-dilatation balloon. The post-dilatation balloon 134 may be inflated to deploy the device 2 and/or further expand the deployed stent against the blood vessel wall, such that the stent conforms to the blood vessel wall.

FIG. 13B illustrates a method for utilizing the delivery apparatus 130 of FIG. 13A to implant the device 2. An optional introducer sheath 138 may be first inserted into the patient over a guidewire. Once the introducer sheath 138 is in position, the guidewire is withdrawn from the lumen of the introducer sheath. The delivery catheter 136, which carries the stent/filter 2, the pre-dilatation balloon 132, and the post-dilatation balloon 134, can then be inserted into the patient's body through the introducer sheath 138. Once the distal portion of the delivery catheter is positioned over a stenosed region 34 in the blood vessel 52, the user holds the two balloons 132, 134 and the device 2 in place, and partially retracts the delivery catheter 136 in the proximal direction to expose the distal portion 140 of the stent/filter 2. The post-dilatation balloon 134 is then partially inflated to expand the distal portion of the stent/filter 2. With the distal portion of the stent/filter 2 expanded to serve as a filter for distal protection, the post-dilatation balloon 134 may be deflated to facilitate blood flow through the filter. The user may also leave the post-dilatation balloon 134 partially inflated while proceeding with the following steps.

The delivery catheter 136 is further retracted to expose the proximal portion of the stent/filter 2 and the pre-dilatation balloon 132. FIG. 13C shows the post-dilatation balloon 134 deflated and the delivery catheter 136 retracted to expose the pre-dilatation balloon 132. The pre-dilatation balloon 132 is then inflated to dilate the stenosed region 34. The pre-dilatation balloon 132 may be inflated and then deflated through several inflation-deflation cycle to facilitate the breaking apart and/or removal of tissues/particles (e.g., plaque, etc.) that has built up over the stenosed region 34. Debris broken off the stenosed region is captured by the expanded portion of the device 2.

Once the pre-dilatation process is completed, the user retracts the pre-dilatation balloon 132 into the lumen of the delivery catheter 136. Optionally, prior to retracting the pre-dilatation balloon 132, the user may apply suction through the delivery catheter 136 to remove debris captured by the distal portion 140 of the device 2. With the pre-dilatation balloon 132 withdrawn into the lumen of the delivery catheter 136, the post-dilatation balloon 134 is expanded to deploy the proximal portion of the device 2. Once the stent/filter 2 is fully deployed, the user may further inflate the post-dilatation balloon 134 to further expand the stent/filter 2 and force the stent/filter 2 to conform to the vessel wall 52. In certain conditions, it may be desirable to inflate and deflate the post-dilatation balloon 134 through several inflation-deflation cycles to improve the expansion of the stent/filter 2 over the stenosed region 34. With the stent/filter 2 fully deployed over the stenosed region 34, the user retracts the post-dilatation balloon 134 into the lumen of the delivery catheter 136. The delivery catheter 136 along with the two balloons 132, 134 is then removed from the patient's body.

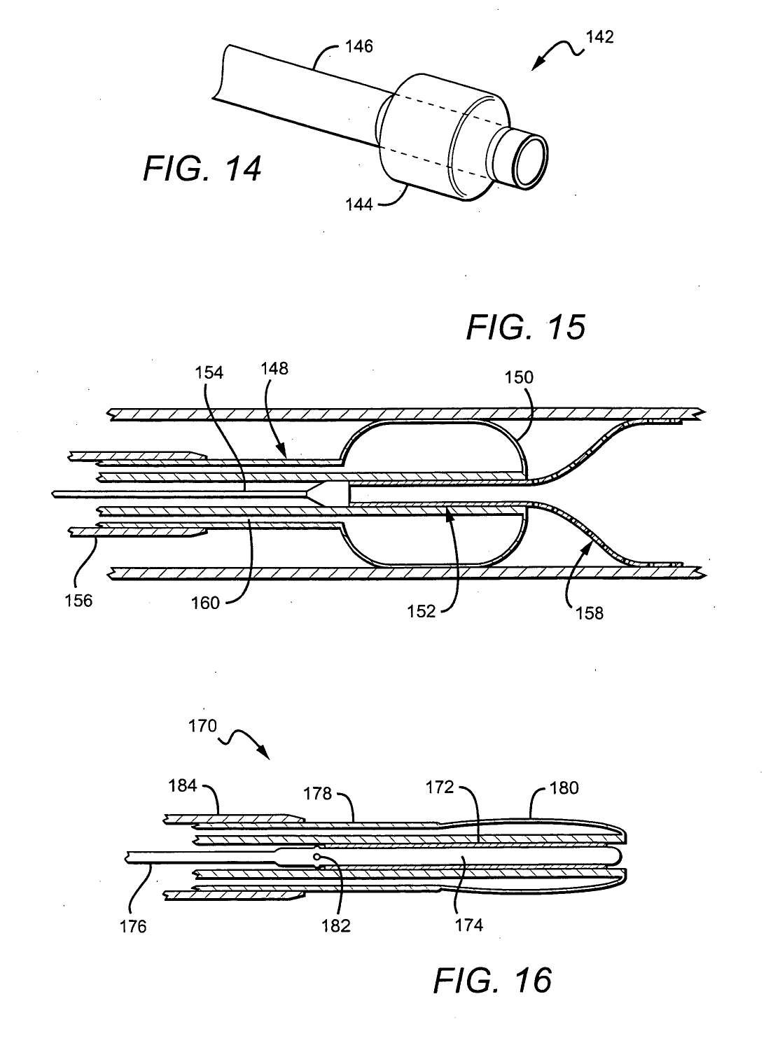

In another variation, a catheter 142 with a balloon 144 integrated on the circumferential surface of the elongated catheter body 146 is utilized to provide a conduit into the patient's body. The catheter 142 is configured with a first lumen extending from the distal end to the proximal end to serve as a conduit for introducing a device into the patient's body. A second lumen is provided within the catheter body to serve as a fluid conduit for inflating a balloon positioned over the shaft of the catheter body. The balloon may comprise a compliant material, a non-compliant material, or a combination thereof. In one variation, the catheter 142 includes a single balloon 144 positioned around the circumference of the catheter shaft 146, as shown in FIG. 14. The balloon may be positioned anywhere along the length of the catheter shaft. For example, the balloon may be positioned at the distal end of the catheter. In another variation, the catheter is configured with two or more balloons positioned over the circumferential surface of the catheter body.

FIG. 15 illustrates an exemplary method for utilizing a delivery catheter with an integrated balloon to deliver a stent 152. In this example, the delivery catheter 148 includes an elongated catheter body with coaxial lumens, and a balloon 150 positioned at the distal portion of the catheter body. A self-expandable stent 152 is placed within the lumen of the delivery catheter 148. A pusher-wire 154 is slidably positioned within the delivery catheter 148 lumen proximal to the stent 152. The delivery catheter 148, loaded with the stent 152, is then inserted into the patient's body through an introducer sheath 156. With the distal portion of the delivery catheter 148 positioned over the intended stent placement site, the user holds the pusher-wire 154 in place, and retracts the delivery catheter 148 in the proximal direction to expose the distal portion 158 of the stent 152. The distal portion 158 of the stent 152 expands and forms a filter at the distal end of the delivery catheter 148. The balloon 150 on the delivery catheter 148 is then inflated by injecting a fluid into the outer lumen 160 of the delivery catheter 148, as shown in FIG. 15. The inflated balloon dilates the section of the vessel surrounding the balloon. As discussed earlier, in certain applications, it may be desirable to inflate and then deflate the balloon through several inflation-deflation cycles. Debris that broke off the vessel wall due to the dilation of the vessel is captured by the proximal portion 158 of the stent 152, which is positioned down stream from the dilation site. Once the dilatation process is completed, the delivery catheter 148 is further retracted in the proximal direction to deploy the proximal portion of the stent 152.

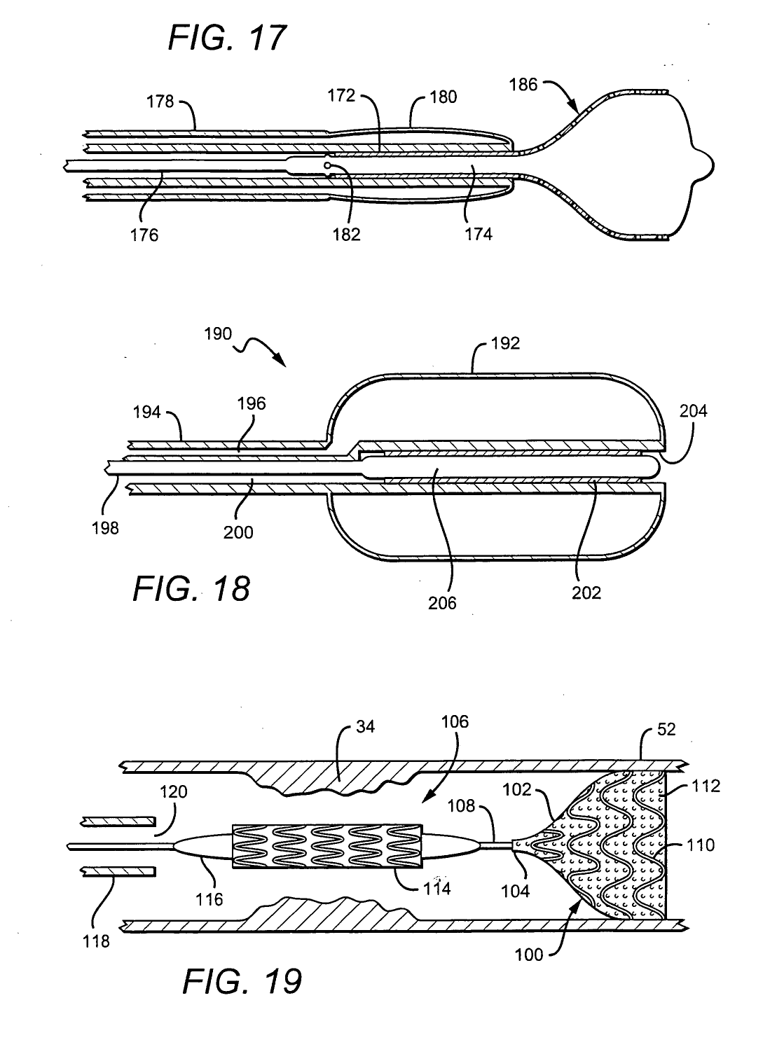

FIG. 16 illustrates another variation of a stent deployment apparatus 170. In this variation, the deployment apparatus 170 includes a stent 172 positioned over the balloon 174 on a balloon catheter 176. Optional raised profiles 182 may be provided on the proximal portion of the balloon 180 to prevent the stent 172 from migrating. The balloon catheter 176 and stent 172 unit is placed within the lumen of a delivery catheter 178 with an integrated balloon 180, as shown in FIG. 16, for insertion into a patient's body. An optional delivery sheath may be utilized to assist with the introduction of the deployment apparatus 170. With the distal portion of the delivery catheter 178 positioned over the stenosed region, the user can retract the catheter 178 to expose the distal portion 186 of the stent 172. If the stent 172 is self-expandable, the distal portion 186 of the stent 172 will expand and engage the wall of the blood vessel. Otherwise, the balloon 174 on the balloon catheter 176 may be partially inflated to expand the distal portion 186 of the stent 172, as shown in FIG. 17. Once the distal portion 186 of the stent has expanded to serve as the filter, the balloon 174 on the balloon catheter 176 may be deflated to facilitate blood flow through the expanded portion of the stent. With the distal portion of the stent expanded to provide distal protection, the balloon 180 on the circumferential surface of the delivery catheter 178 is inflated to dilate the blood vessel. After the completion of the pre-dilatation process, the balloon 180 on the delivery catheter 178 is deflated, and the delivery catheter 178 is further retracted to deploy the rest of the stent 172. If the stent 172 is self-expandable, it will deploy once the compression force from the delivery catheter 178 is removed. The balloon 174 on the balloon catheter 176 can also be inflated to assist with the expansion of the stent 172. Once the stent 172 is deployed, the balloon 174 on the balloon catheter 176 may be further inflated, such that the stent 172 is further expanded to conform to the vessel wall. With the stent 172 fully deployed, the balloon 174 on the balloon catheter 176 is deflated and withdrawn from the patient's body along with the delivery catheter 178.

FIG. 18 illustrates another variation of a delivery catheter 190 with an integrated balloon 192. In this variation, the delivery catheter 190 can include an elongated dual-lumen catheter body 194. The distal portion of the catheter includes a chamber 204 which may house a medical device for delivery. In FIG. 18, the delivery catheter 190 is shown with the balloon 192 inflated due to fluids infused through the first lumen 196 of the delivery catheter 190. A balloon catheter 198 is slidably positioned within the second lumen 200 of the delivery catheter 190. A compressed stent 202, positioned over the deflated balloon 206 on the balloon catheter 196, is placed in the chamber 204 within the distal end of the delivery catheter 190.

It is noted that the delivery apparatus disclosed herein may also be utilized to deliver a traditional stent, a coated stent, a polymer layer covered stent, and various other stent configurations, and the deployment methods disclosed herein may be utilized to deploy various medical devices.

In another aspect of the invention, shown herein is FIG. 19, a retrievable filter, is provided at the distal end of a stent deployment apparatus. In one variation, the filter includes a collapsible cone-shaped filter 100 with the tip of the cone 102 attached to the distal end 104 of the deployment apparatus 106, while the filter 100 is configured to expand radially proximate the distal portion. In another variation of the deployment apparatus, the filter may include an expandable stent structure having pores for filtering, and one end of the stent structure is attached to the tip of a guidewire type rod.

As shown in FIG. 19, the deployment apparatus 106 may include a balloon 108 catheter having a cone-shaped filter 100 attached to the distal end 104 of the catheter 108. The filter 100 may have a self-expanding wire mesh 110 (e.g., nitinol, etc.) forming the collapsible filter's structure, and a porous polymeric layer 112 positioned over the self-expanding wire mesh 110. A stent 114 is positioned over the balloon 116 on the balloon catheter 108. The stent 114 may include either a self-expandable or a balloon-expandable material. In one deployment example, the deployment apparatus 106 is loaded with the stent 114 and then placed within the lumen of a delivery catheter 118. The delivery catheter 118 loaded with the deployment apparatus 106 is inserted into the patient's circulatory system through an introducer sheath. The tip of the delivery catheter is inserted through the stenosed region 34. While holding the deployment apparatus 106 in place, the delivery catheter 118 is retracted to deploy the filter 100 at a location distal to the stenosed region 34. Further retraction of the delivery catheter 118 exposes the stent 114 for deployment. The balloon 116 may then be inflated to expand the stent and the stenosed region 34.

Once the stent 114 is deployed, the balloon 116 is deflated and suction may be applied through the introducer sheath or the delivery catheter 118 to remove any debris captured by the deployed filter 100. Once the debris has been removed, the delivery catheter 118 is advanced in the distal direction to collapse the filter 100. Since the filter 100 is configured in a reversed-cone configuration, the opening 120 of the delivery catheter 118 can slide over the circumferential surface of the cone-shaped filter 100 and force the expanded filter 100 to collapse towards a longitudinal axis. Once the filter 100 is captured within the lumen of the delivery catheter 118, the delivery catheter 118 along with the introducer sheath may be removed from the patient's body. In one design variation, the delivery catheter is configured with an integrated balloon on the outer circumferential surface, such that the stenosed region 34 can be pre-dilated before the stent 114 is deployed over the stenosed region 34.

In another variation, the delivery apparatus may include a flexible rod with a collapsible cone-shaped filter attached to the distal end of the rod. A self-expandable stent is then positioned over the shaft of the flexible rod. The apparatus is inserted into the patient with a delivery catheter in a manner similar to that described above. As the delivery catheter is retracted in the proximal direction, the filter deploys first, followed by the deployment of the stent. Once the stent has expanded, a balloon catheter may be introduced into the lumen of the stent to further expand the stent along with the stenosed region. After the expansion procedure, the balloon catheter is removed and suction may be applied to remove any debris. The user then advances the delivery catheter distally to capture the cone shaped filter and removed it from the patient's body. In the above procedure, the balloon catheter may be introduced through the delivery catheter if the compressed balloon is small enough. In another approach, the delivery catheter is removed prior to the insertion of the balloon catheter into the lumen of the introducer sheath. Once the stent is deployed and the balloon is removed from the introducer sheath, the delivery catheter is inserted to remove the filter.

In another variation of the procedure, a flexible rod with a cone-shaped filter at the distal end is inserted inside a delivery catheter to deploy the cone-shaped filter at a location distal the stenosed region. Once the filter is deployed, the user can remove the delivery catheter and leave the filter along with its shaft in the blood vessel. A balloon catheter is then introduced to expand the stenosed region. With the stenosed region expanded, the balloon catheter is removed and a delivery system carrying a stent is inserted to deploy the stent over the stenosed region. Once the stent is deployed, the delivery catheter is inserted again. Suction may be applied through the delivery catheter to remove any particles capered by the filter. In another variation, the suction may be applied through the introducer sheath is one is utilized in the procedure. Once the debris has been removed, the introducer catheter can be utilized to collapse and remove the filter.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually set forth herein.

What is claimed is:

1. A vascular intervention device, comprising:
a stent; and
a filter coupled to a distal portion of said stent.

2. The vascular intervention device according to claim 1, wherein said filter comprises a plurality of pores positioned over the distal portion of said stent for filtering particles within blood flow.

3. The vascular intervention device according to claim 2, wherein said stent is comprised of a self-expanding material.

4. The vascular intervention device according to claim 1, further comprising a polymeric layer surrounding a circumferential surface of said stent, the polymeric layer having a plurality of pores in a distal end thereof.

5. The vascular intervention device according to claim 4, wherein the polymeric layer comprises ePTFE.

6. The vascular intervention device according to claim 4, wherein said polymeric layer comprises one or more materials selected from a group consisting of ePTFE urethane, silicone, or nylon, and combination thereof.

7. The vascular intervention device according to claim 4, wherein said polymeric layer further comprises a therapeutic agent.

8. The vascular intervention device according to claim 4, wherein said polymeric layer further comprises a biological material.

9. The vascular intention device according to claim 5, wherein said stent comprises nitinol.

10. The vascular intervention device according to claim 1, further comprising a polymeric layer surrounding a circumferential surface of said stent, said polymeric layer comprising a first set of pores positioned over the distal portion of said stent and a second set of pores positioned over a proximal portion of said stent, wherein said first set of pores are smaller than said second set of pores.

11. The vascular intervention device according to claim 10, wherein each of said first set of pores has a diameter from about 50 micron to about 100 micron.

12. The vascular intervention device according to claim 10, wherein each of said second set of pores has a diameter from about 20 micron to about 200 micron.

13. The vascular intervention device according to claim 10, wherein said polymeric layer comprises ePTFE.

14. The vascular intervention device according to claim 10, wherein said stent is self-expandable.

15. The vascular intervention device according to claim 10, further comprises an expansion mechanism having a portion positioned within a lumen of the stent.

16. The vascular intervention device according to claim 15, wherein the expansion mechanism comprises a balloon configured to expand the stent.

17. The vascular intervention device according to claim 1, further comprising a polymeric layer surrounding a distal circumferential surface of said stent, said polymeric layer comprising a plurality of pores.

18. The vascular intervention device according to claim 1, wherein said filter comprises a collapsible porous structure positioned over a distal opening of said stent.

19. The vascular intervention device according to claim 18, wherein said collapsible porous structure comprises a biodegradable material.

20. The vascular intervention device according to claim 18, wherein said collapsible porous structure is configured to be dismantled after the stent has been deployed within a blood vessel.

21. The vascular intervention device according to claim 1, wherein said filter is positioned over a distal opening of said stent.

22. The vascular intervention device according to claim 1, wherein said filter comprises a porous film positioned over a distal opening of said stent.

23. The vascular intervention device according to claim 1, wherein said filter comprises a cone shaped structure positioned over a distal opening of said stent.

24. The vascular intervention device according to claim 1, wherein said filter is positioned within a lumen of said stent.

25. The vascular intervention device according to claim 24, wherein said filter comprises a porous film.

26. A method of implanting a stent within a blood vessel, comprising:
inserting said stent into the blood vessel, wherein a distal portion of said stent comprises a filter; and
deploying the distal portion of said stent at a location distal of a stenosed region to capture particles breaking from said stenosed region while permitting fluids to pass through.

27. The method of implanting a stent according to claim 26, wherein the distal portion of said stent in the deploying step comprises a plurality of pores to allow blood to flow through.

28. The method according to claim 26, further comprising the step of deploying a proximal portion of said stent over said stenosed region.

29. The method according to claim 28, further comprising the step of expanding said stent and forcing at least a proximal portion of said stent against an inner wall of said blood vessel.

30. The method according to claim 29 wherein said filter comprises a polymeric layer covering at least the distal portion of said stent, wherein said polymeric layer comprises a plurality of pores, and each of said pores is at least 50 micron in diameter.

31. The method according to claim 26, wherein the distal portion of said stent in the deploying step comprises a polymeric layer covering a circumferential surface of said stent, and said polymeric film includes a plurality of pores.

32. The method according to claim 26, wherein said stent in the deploying step comprises a plurality of pores located over the distal portion of said stent forming said filter.

33. The method according to claim 26, wherein said stent in the deploying step further comprises a material woven over at least the distal portion of the stent.

34. The method according to claim 32, wherein each of said plurality of pores has a diameter between about 50 micron to about 100 micron.

35. The method according to claim 26, further comprising the step of expanding an expandable porous surface to form said filter.

36. The method according to claim 26, wherein said deploying step further comprises allowing the distal portion of said stent to expand and engage an inner wall of the blood vessel while keeping a proximal portion of said stent compressed.

37. The method according to claim 36, further comprising the step of deploying a proximal portion of said stent in said stenosed region.

38. The method according to claim 26, wherein the inserting said stent step further comprises inserting an elongated body, which houses said stent within a distal portion of said elongated body, into said blood vessel, wherein said elongated body further comprises a first mechanism for dilating a segment of the blood vessel prior to deployment of a proximal portion of said stent, and a second mechanism for deploying said stent.

39. The method according to claim 26, further comprising the step of dilating the stenosed region.

40. The method according to claim 39, further comprising the step of deploying a proximal portion of said stent.

41. The method according to claim 40, further comprising the step of expanding said stent and forcing said stent against an inner wall of the blood vessel.

42. The method according to claim 41, wherein the inserting step further comprises inserting an elongated body, which houses said stent in a distal portion of said elongated body, into the blood vessel, wherein mechanisms for accomplishing the deploying the distal portion step, the dilating step, the deploying the proximal portion step, and the expanding step, are all housed within said elongated body.

43. An implantable device comprising:
a tubular structure configured for expanding a stenosed region in a blood vessel; and
means for filtering blood flow.

44. A vascular intervention apparatus comprises:
a stent, wherein a distal portion of said stent is configured as a filter;
means for dilating a segment of a blood vessel prior to deployment of said stent; and
means for deploying said stent.
